(19) United States
(12) Patent Application Publication
(10) Pub. No.: US 2005/0215982 A1
(43) Pub. Date: Sep. 29, 2005

(54) RF medical device

(76) Inventors: Luis Malave, Salem, MA (US); Marc Vogt, Rye, NH (US); Mohsen Moghaddami, Londonderry, NH (US); Paul Wolejko, Newburyport, MA (US); Richard Aparo, West Newton, MA (US); Adam Casey, Abington, MA (US); Charles Cox, Cambridge, MA (US); Stuart Perry, Wellesley Hills, MA (US); Calvert Hawkes, Sarasota, FL (US)

Correspondence Address: INSULET CORPORATION, 9 Oak Park Drive, Bedford, MA, 01730, US

(21) Appl. No.: 10/835,727

(22) Filed: Apr. 30, 2004

Related U.S. Application Data
(60) Provisional application No. 60/466,708, filed on Apr. 30, 2003.
(60) Provisional application No. 60/466,704, filed on Apr. 30, 2003.
(60) Provisional application No. 60/466,589, filed on Apr. 30, 2003.

(51) Int. Cl. A61K 9/22

(52) U.S. Cl. 604/890.1; 128/903; 128/904

(57) ABSTRACT

A system for delivering a fluid to a patient includes a remote controller and an infusion pump. The infusion pump includes a dispenser for dispensing the fluid. An RF telemetry portion is configured to receive an RF data signal, from the remote controller, that is broadcast in a non-restricted frequency band. A processing portion is configured to process the RF data signal received by the RF telemetry portion, and control the dispenser in accordance with the RF data signal received by the RF telemetry portion.

### RELATED APPLICATIONS

This application claims the priority of the following applications, each of which is herein incorporated by reference: U.S. Provisional Application Ser. No.: 60/466708, entitled “Infusion Device System Hardware and Method of Using The Same”, filed 30 Apr. 2003; U.S. Provisional Application Ser. No.: 60/466704, entitled “Infusion Device System Programming and Method of Operating an Infusion Device”, filed 30 Apr. 2003; and U.S. Provisional Application Ser. No.: 60/466589, entitled “Remote Communications Methods for Infusion Devices”, and filed 30 Apr. 2003.

### FIELD OF THE INVENTION

This invention relates to medical devices/systems and, more particularly, to medical devices/system having RF communication capabilities.

### BACKGROUND

Ambulatory infusion devices/pumps were developed to deliver liquid medicaments to patients. Typically, infusion devices are capable of providing sophisticated fluid delivery profiles (e.g., bolus doses, continuous basal infusions, variable flow delivery rates, etc.) and often automate the delivery of insulin when treating diabetes.

Currently available ambulatory infusion devices are typically bulky, heavy, expensive and fragile. Additionally, these devices are typically difficult to program and prepare for infusion. Further, filling these devices with the medicament can be difficult and often requires that the user carry both the medicament and the filling accessories. Often, these devices require specialized care, maintenance, and cleaning to assure proper functionality and safety for their intended long term use. Unfortunately, as these devices tend to be expensive, healthcare providers typically limit the patient populations to which these devices are made available.

### SUMMARY OF THE INVENTION

According to an aspect of this invention, a system for delivering a fluid to a patient includes a remote controller and an infusion pump. The infusion pump includes a dispenser for dispensing the fluid. An RF telemetry portion is configured to receive an RF data signal, from the remote controller, that is broadcast in a non-restricted frequency band. A processing portion is configured to process the RF data signal received by the RF telemetry portion, and control the dispenser in accordance with the RF data signal received by the RF telemetry portion.

One or more of the following features may also be included. The non-restricted frequency band may be 13.40-13.70 megahertz. The RF telemetry portion may be configured to receive and/or transmit data encoded within a 13.56 megahertz carrier signal.

According to another aspect of this invention, a medical device includes an RF telemetry portion configured to receive an RF data signal that is broadcast in a non-restricted frequency band. A processing portion is configured to process the RF data signal received by the RF telemetry portion.

One or more of the following features may also be included. The processing portion may include a main processing unit and an interlock processing unit. The non-restricted frequency band may be 13.40-13.70 megahertz. The RF telemetry portion may be further configured to receive and/or transmit data encoded within a 13.56 megahertz carrier signal.

The RF telemetry portion may include compact antenna, such as a spirally-wound antenna or a helically-wound antenna. The effective length of the compact antenna may be a defined percentage of a wavelength of a carrier signal.

The RF data signal may include a defined validation sequence and the RF telemetry portion may be further configured to examine the RF data signal to confirm that the RF data signal includes the defined validation sequence. The RF telemetry portion may be further configured to transmit an acknowledgement signal to the device transmitting the RF data signal if it may be determined that the RF data signal includes the defined validation sequence.

The RF telemetry portion and at least a first portion of the processing portion may be incorporated into a single microchip, such as an application-specific integrated circuit.

A dispensing apparatus, responsive to the processing portion of the medical device, may dispense medicament (e.g., insulin) in accordance with the RF data signal.

According to another aspect of this invention, a method of communicating with a medical device includes receiving an RF data signal that is broadcast in a non-restricted frequency band, and processing the RF data signal received.

One or more of the following features may also be included. The non-restricted frequency band may be 13.40-13.70 megahertz. Receiving an RF data signal may include receiving data encoded within a 13.56 megahertz carrier signal. Data encoded within a 13.56 megahertz carrier signal may be transmitted.

The RF data signal may include a defined validation sequence, and the RF data signal may be examined to confirm that the RF data signal includes the defined validation sequence. An acknowledgement signal may be transmitted to the device transmitting the RF data signal if it is determined that the RF data signal includes the defined validation sequence.

The above-described method may also be implemented as a sequence of instructions executed by a processor.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic perspective view of a fluid delivery system, including an infusion pump and a remote controller;

FIG. 2 is an isometric top view of the infusion pump of FIG. 1;

FIG. 3 is an isometric bottom view of the infusion pump of FIG. 1;

FIG. 4 is an isometric view of the infusion pump of FIG. 1 (with the upper housing removed); and

FIG. 5 is a front view of the remote controller of FIG. 1; and

FIG. 6 is a diagrammatic view of the infusion pump of FIG. 1.

### DETAILED DESCRIPTION OF THE PREFERRED EMBODIMENTS

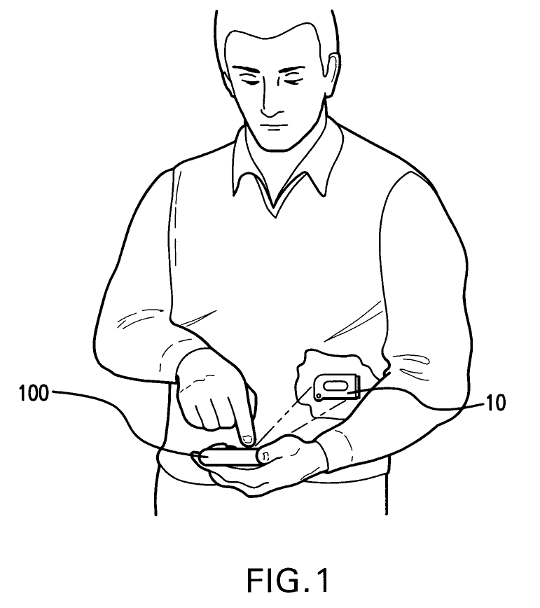

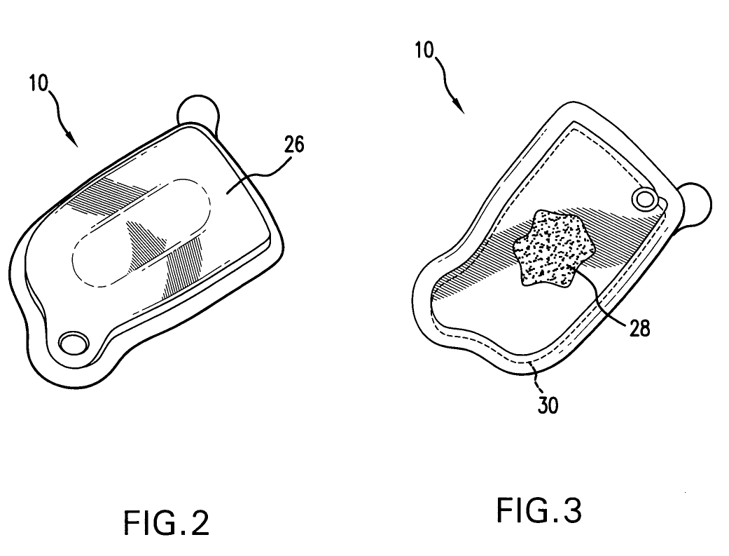

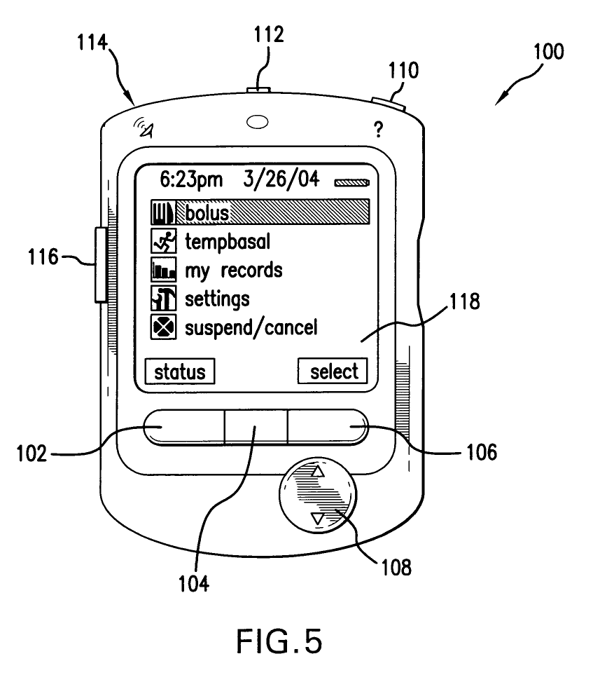

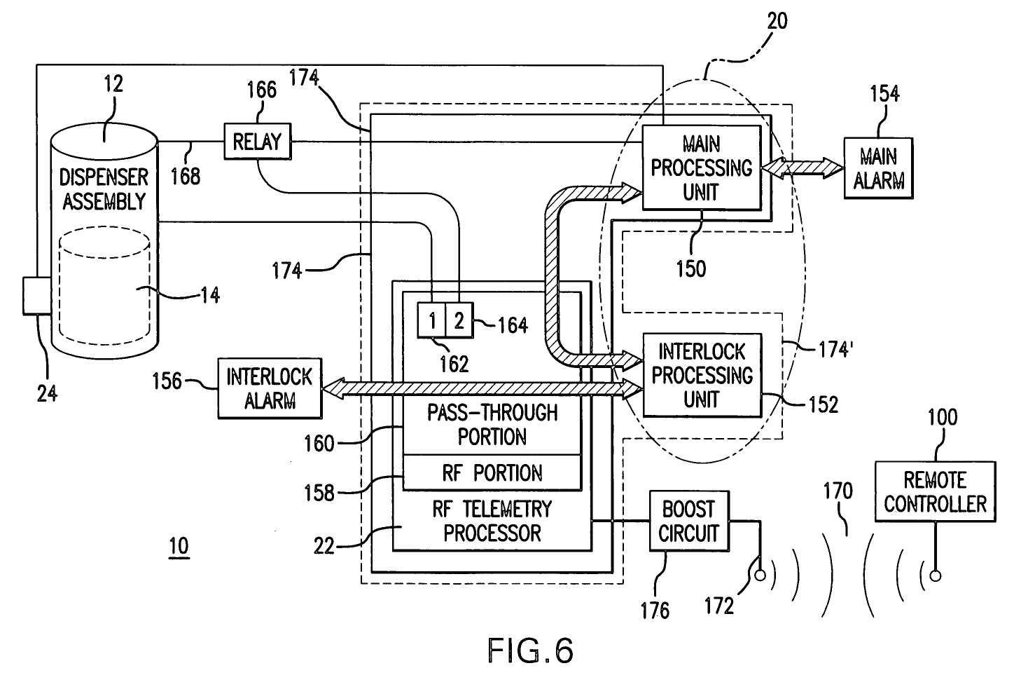

Referring to FIGS. 1-4, there is shown a remotely-controlled, disposable infusion pump 10, which is typically used with remote controller 100 (shown in FIGS. 1 and 5). Examples of similar infusion pumps are disclosed in co-pending U.S. patent application Ser. No. 09/943,992, filed on Aug. 31, 2001, which is herein incorporated by reference. Infusion pump 10 may incorporate a new and improved RF telemetry processor and local processor, which are discussed below in greater detail and shown in FIG. 6.

While the new and improved RF telemetry processor and local processor of the present disclosure are described with reference the exemplary embodiment of infusion pump 10 and remote controller 100, it should be understood that the present disclosure is broadly applicable to any form of programmable infusion pumps. For example, the new and improved RF telemetry processor and local processor of the present disclosure may be used with programmable ambulatory insulin infusion pumps of the sort currently commercially available from a number of manufacturers, including without limitation and by way of example, Medtronic Minimed under the trademark PARADIGM, Animas Corporation under the trademarks IR 1000 and IR 1200, Smiths Medical under the trademark Deltec COZMO, DANA Diabecare USA, and others.

Infusion pump 10 is used to deliver medicaments to a person or animal. The types of medicaments that may be delivered (via infusion pump 10) include, but are not limited to, insulin, antibiotics, nutritional fluids, total parenteral nutrition (i.e., TPN), analgesics, morphine, hormones/hormonal drugs, gene therapy drugs, anticoagulants, analgesics, cardiovascular medications, AZT, or chemotherapeutics, for example. The types of medical conditions that infusion pump 10 may be used to treat include, but are not limited to, diabetes, cardiovascular disease, temporal pain, chronic pain, cancer, AIDS, neurological disease, Alzheimer's Disease, ALS, Hepatitis, Parkinson's Disease or spasticity, for example.

Infusion pump 10 is typically disposable and adapted for attachment to the skin of a patient for infusing a medicament, such as insulin, into the patient on a regular basis. The infusion pump 10 may have a usable life of about 72 hours, for example, before being removed from the patient and discarded.

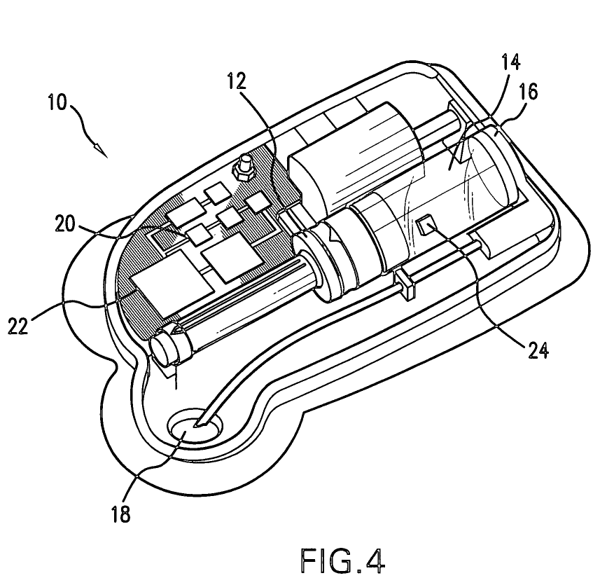

Referring to FIG. 4, infusion pump 10 typically includes a dispenser assembly 12 for causing medicament from fluid reservoir 14 to flow through flow path assembly 16 to transcutaneous access tool (e.g., needle) 18 for infusion into the patient. The volume of reservoir 14 is chosen to best suit the therapeutic application of infusion pump 10, impacted by such factors as the available concentrations of medicament to be delivered, the acceptable time between refill/disposal of infusion pump 10, and size constraints, for example

Local processor 20 (e.g., one or more processors or electronic microcontrollers) is connected to dispenser assembly 12, and is programmed to control the flow of medicament to the transcutaneous access tool 18 based on flow instructions from the separate, remote controller 100 (as shown in FIG. 5). RF telemetry processor 22, which is coupled to local processor 20, receives flow instructions from remote controller 100 and provides them to local processor 20.

Infusion pump 10 typically includes a power supply (e.g., a battery or capacitor; not shown) that supplies power to local processor 20. This power supply may be non-serviceable (e.g., a litium ion battery soldered to a circuit board) or replaceable (e.g., a AAA battery).

As shown in FIG. 4, infusion pump 10 may also include various sensors/transducers, such as a flow condition sensor assembly (not shown) or a fill sensor 24 (to be discussed below in greater detail), that transmit information to local processor 20 concerning the condition and status of infusion pump 10.

Infusion pump 10 includes housing 26, which contains and protects dispenser assembly 12, reservoir 14, flow path assembly 16, transcutaneous access tool 18, local processor 20, and RF telemetry processor 22. Infusion pump 10 may be provided with an adhesive layer 28 (as shown in FIG. 3) on the lower surface 30 of housing 26 for temporarily securing infusion pump 10 directly to the skin of the patient.

As discussed above, infusion pump 10 includes RF telemetry processor 22 that facilitates the programming of local processor 20 via remote controller 100. Commands may be transmitted between infusion pump 10 and remote controller 100 via a communication circuit (not shown) incorporated into remote controller 100.

The outer surfaces of housing 26 are typically free of any user input components (e.g., buttons/interfaces/electromechanical switches) that would allow the user to program local processor 20), thus reducing the size, complexity and cost of infusion pump 10. Alternatively, infusion pump 10 may include an integrated user interface (not shown) with some or all of the features of remote controller 100, thus allowing the user to directly input instructions/commands to infusion pump 10.

Remote controller 100 typically includes: user input components that allow the user to provide information; user output components that allow the user to receive information; a processor (hereinafter referred to as the “remote” processor) coupled to the user input components and the user output components and configured to provide instructions to the infusion pump; and one or more computer programs that provide instructions to the remote processor.

The computer programs instruct the remote processor to receive information from the user via the user input components, provide information to the user via the user output components, and provide instructions/commands to infusion pump 10.

As shown in FIG. 5, the user input components may include: electromechanical switches, such as three soft key selection switches 102, 104, 106; an up/down navigation toggle switch 108; a “display user information” switch 110; a power on/off switch 112; a “check pump status” switch 114; and an “instant bolus” switch 116. The user output components may include: a visual display (e.g., LCD screen 118); a sound making device (e.g., a buzzer; not shown); and/or a vibrating element (not shown).

Soft key selection switches 102, 104, 106 cause remote controller 100 to perform the action indicated by the label (on LCD screen 118) above the switch in question. If there is no label above one of the switches 102, 104, 106, pressing the switch at that time will result in no activity. The up/down navigation toggle switch 108 is used to navigate a menu, enter a number, or change a character during text entry.

LCD screen 118 displays icons to distinguish between various features. For non-menu pages, the icon may be displayed in the upper-left corner of LCD screen 118. On menu pages, the icon may be displayed to the left of the currently highlighted menu item, except on the main menu where an icon is displayed to the left of all menu items.

System functions are navigated via menus, which list the functions available to the user and allow the user to quickly enable the appropriate function. These menus consist of a set of options in a list, with a highlight that moves up and down in response to the up/down navigation toggle switch 108. When the highlight is over the appropriate option, the user depresses one of the three soft key selection switches 102, 104, 106 to select the option. Text entry in the system is accomplished via the soft keys 102, 104, 106 and the up/down toggle switch 108. The user moves the flashing up/down icon left and right using two of the soft keys, and changes the character above the icon using the up/down navigation toggle switch 108. Pressing the up/down toggle switch 108 changes the letter to the next letter in the sequence.

Although not shown, remote controller 100 may include additional components such as an integrated glucose meter (e.g., a TheraSense® FreeStyle™ Glucose Meter that is available from Abbott Diabetes Care of Alameda, Calif.). If such additional components are includes, the user interface components of remote controller 100 are typically configured to operate the additional components.

According to one embodiment, RF telemetry processor 22 of infusion pump 10 receives electronic communication from remote controller 100 using radio frequency or other wireless communication standards/protocols. In a preferred embodiment, RF telemetry processor 22 is a bidirectional communication device, that includes a receiver portion and a transmitter portion. This, in turn, allows infusion pump 10 to transmit information to remote controller 100. In this embodiment, remote controller 100 is also capable of bidirectional communication, thus allowing remote controller 100 to receive the information sent by infusion pump 10.

Local processor 20 of infusion pump 10 typically includes all of the computer programs and electronic circuitry needed to allow a user to program local processor 20. Such circuitry may include one or more microprocessors, digital and/or analog integrated circuits, and other various passive and active electronic components, for example.

As will be discussed below in greater detail, local processor 20 also typically includes the programming, electronic circuitry and memory to activate dispenser assembly 12 at the programmed time intervals. In a preferred embodiment, user instructions/commands are processed in remote controller 100 to generate one or more specific flow control instructions, (i.e., drive signals) for infusion pump 10. Alternatively, the user may input the instructions/commands into remote controller 100, such that the instructions/commands are transmitted from remote controller 100 to infusion pump 10, where the instructions/commands are processed to generate the flow control instructions (i.e., drive signals) for infusion pump 10.

Referring to FIG. 6, local processor 20 typically includes main processing unit 150 and interlock processing unit 152. Additionally, infusion pump 10 typically also includes main alarm unit 154, interlock alarm unit 156, RF telemetry processing unit 22 (which includes RF (i.e., radio frequency) portion 158 and a pass-through portion 160).

In order to conserve battery power, several of the components of infusion pump 10 are maintained in a “sleep” mode that reduces power consumption. RF portion 158 of RF telemetry processing unit 22 “wakes up” at predefined intervals (e.g., every 125 milliseconds) and polls a defined frequency (e.g., 13.56 megahertz) to determine if remote controller 100 is trying to communicate with infusion pump 10. If data packets are not available for receipt, RF portion 158 of the RF telemetry processing unit 22 returns to “sleep” mode for the predefined interval.

However, if a data packet is available for receipt, RF portion 158 receives the data packet and examines it to verify that the packet was received from an authorized source. Typically, this verification is performed by examining the content of the data packet received to see if it contains a defined bit signature/validation sequence (e.g., 0110 0110, or 1001 1001). If present, RF portion 158 transmits an acknowledgement signal to remote controller 100 that requests transmission of the instruction set. Additionally, RF portion 158 may verify that the data packet received is valid, which may be determined using, for example, a checksum.

At this point, RF portion 158 “wakes up” main processing unit 150 and the data packets received are provided to main processing unit 150 for further examination and processing. Typically, “wake up” signals are transmitted between communicating devices (e.g., main processing unit 150, interlock processing unit 152, and RF telemetry processing unit 22, for example) via the various buses (not shown) that interconnect the communicating devices.

Main processing unit 150 may reexamine the received data packet(s) to verify that infusion pump 10 is truly the intended recipient of the data packet. As discussed above, one or more of the data packets received typically includes a unique bit signature/validation sequence that identifies the intended recipient of the data packet. If the unique bit signature/validation sequence within the packet does not match the unique bit signature/validation sequence of infusion pump 10, infusion pump 10 is not the intended recipient, the data packet is rejected by main processing unit 150, and the main processing unit 150 notifies the RF portion 158 of the RF telemetry processing unit 22 that the data packet received was misdirected.

However, if infusion pump 10 is indeed the intended recipient of the data packet, main processing unit 150 accepts the data packet, as the received data packet is a portion of a valid instruction set being transmitted by remote controller 100. This packet receipt and examination process continues for subsequently-received data packets until the instruction set received is complete. Once received, the complete instruction set includes a main instruction portion (for the main processing unit 150) and an interlock instruction portion (for the interlock processing unit 152).

Once a complete instruction set is received, main processing unit 150 wakes up interlock processing unit 152 so that the interlock portion of the received instruction set can be transferred to interlock processing unit 152. Typically, each data packet received includes an interlock portion and a main portion (in addition to the identification information described above). The interlock portion (for use by interlock processing unit 152) typically includes instructions in terms of pulses of medicament (e.g., insulin) per unit time (e.g., per half hour). The main portion (for use by main processing unit 150) typically includes instructions in terms of the number of partial pulses of medicament (e.g., insulin), and the delay between each partial pulse.

As stated above, RF telemetry processing unit 22 includes pass-through portion 160 that allows for pass-through communications between main processing unit 150 and interlock processing unit 152, and between interlock processing unit 152 and interlock alarm unit 156. As will be discussed below, pass-through portion 160 of RF telemetry processing unit 22 acts as a conduit that completes a circuit between the communicating devices, in that RF portion 158 of RF telemetry processing unit 22 is isolated from and does not modify the signals passed between the communicating devices.

Additionally, pass-through portion 160 of RF telemetry processing unit 22 includes status registers 162, 164 that are readable and writable by devices external to RF telemetry processing unit 22. As will be discussed below, status registers 162, 164 included in RF telemetry processing unit 22 allow main and interlock processing units 150, 152 to confirm the operation of dispenser assembly 12 and, in the event of a failure, prevent the pump drive signals from reaching dispenser assembly 12.

As stated above, once a complete instruction set is received, the interlock portion of the instruction set is transferred to interlock processing unit 152. In the event that interlock processing unit 152 does not acknowledge receipt of the interlock portion of the instruction set, main processing unit 150 assumes that interlock processing unit 152 is malfunctioning and initiates an alarm on main alarm unit 154.

Interlock processing unit 152 and main processing unit 150 are typically powered by separate power supplies (e.g., batteries or capacitors; not shown), are synchronized using a common clock (not shown), and each independently execute their received instruction sets, resulting in a level of redundancy.

Often, a received instruction set will specify that a defined dose of medicament be dispensed at predefined intervals (e.g., ten minutes). At the expiration of one of these predefined intervals, main processing unit 150 contacts (via pass-through portion 160 of RF telemetry processing unit 22) interlock processing unit 152 to confirm that it is the proper time for dispensing the defined dose of medicament. If interlock processing unit 152 fails to respond, main processing unit 150 assumes that interlock processing unit 152 is malfunctioning and initiates an alarm on main alarm unit 154.

Further, in the event that interlock processing unit 152 does not agree that it is the proper time to dispense the defined dose of medicament, interlock processing unit 152 may initiate an alarm on interlock alarm unit 156, via pass-through portion 160 of RF telemetry processing unit 22. Additionally and/or alternatively, main processing unit 150 may initiate an alarm on main alarm unit 154.

If both interlock processing unit 152 and main processing unit 150 concur that it is time to dispense the defined dose of medicament, main processing unit 150 provides the appropriate “pump drive signal” to dispenser assembly 12.

After dispenser assembly 12 completes dispensing the medicament, a completion signal is provided by dispenser assembly 12 to status register 162 to confirm that the medicament was successfully dispensed. Main processing unit 150 and interlock processing unit 152 monitor status register 162 to determine if the medicament was dispensed. If, after a defined period of time (e.g., 1-5 seconds), status register 162 fails to indicate that the medicament was dispensed, main processing unit 150 assumes that dispenser assembly 12 is malfunctioning and main processing unit 150 typically initiates an alarm on main alarm unit 154. Additionally and/or alternatively, interlock processing unit 152 may initiate an alarm on interlock alarm unit 156 (via pass-through portion 160 of RF telemetry processing unit 22).

In addition to the alarms, in the event that dispenser assembly 12 fails to dispense the medicament, the main and/or interlock processing units 150, 152 may provide a dispenser failure signal to a second status register 164. The value of register 164 determines whether a relay 166 (e.g., a FET transistor) that is in the signal line 168 that provides the “pump drive signal” to dispenser assembly 12 is energized. Accordingly, in the event that the dispenser assembly 12 fails to dispense the defined medicament dose, dispenser assembly 12 is electrically disconnected from the signal line 168 controlling dispenser assembly 12.

When RF telemetry processing unit 22 and remote controller 100 communicate by transmitting an RF data signal across wireless communication channel 170, this communication typically occurs across a non-restricted frequency band, which is a frequency band that is dedicated to public use and not restricted for use by only a certain class of devices. For example, a restricted frequency band is 408-412 megahertz, which is reserved in the United States for the exclusive use of medical devices. An example of a non-restricted frequency band is 13.40-13.70 megahertz, which is dedicated for public use worldwide and has no use device-class restrictions. Specifically, RF telemetry processing unit 22 and remote controller 100 typically communicate using a 13.56 megahertz carrier signal, onto which the individual data packets within the instruction set are encoded.

RF telemetry processing unit 22 is electrically coupled to antenna assembly 172, which facilitates wireless communication with remote controller 100. As it is desirable to minimize the size of infusion pump 10, antenna 172 is typically a compact antenna design (e.g., a spirally-wound antenna or a helically-wound antenna). As is known in the art, it is desirable for the effective length of antenna 172 to be a defined percentage (e.g., 25%, 50% or 100%) of the wavelength of the carrier signal. For a carrier signal of 13.56 megahertz, the wavelength of the carrier signal is 22.100 meters and, therefore, the defined percentages are 5.525 meters, 11.050 meters, and 22.100 meters, respectively.

Since it is desirable to reduce the physical size of infusion pump 10, main processing unit 150 and RF telemetry processing unit 22 are typically incorporated into a single microchip 174, such as an ASIC (i.e., application specific integrated circuit). If main processing unit 150 and RF telemetry processing unit 22 are incorporated into a single microchip, two separate power supplies (not shown) may be required to power the microchip, a first power supply for main processing unit 150 and a second power supply for RF telemetry processing unit 22. Alternatively or additionally, it may be desirable to incorporate interlock processing unit 152, RF telemetry processing unit 22, and main processing unit 150 into a single microchip 174′ (shown in phantom). Since, by design, main processing unit 150 and interlock processing unit 152 are powered by separate power supplies, if all three processing units 150, 152, 22 are incorporated into a single microchip, three power supplies may be required to power microchip 174′.

When incorporating two of more processing units (e.g., main processing unit 150, interlock processing unit 152, and/or RF telemetry processing unit 22) within a single microchip 174, it may be desirable to locate antenna 172 outside of microchip 174, thus reducing the risk of electromagnetic interference within microchip 174. Further, if RF telemetry processing unit 22 includes a boost circuit 176 (i.e., to boost the amplitude of the signal broadcast or received by antenna 172), it may be desirable to also locate boost circuit 176 external to microchip 174 in order to shield main processing unit 150 and/or interlock processing unit 152 from electromagnetic interference.

Dispenser assembly 12 typically includes a fill sensor 24 (e.g., a normally open mechanical switch) that provide an initialization signal to local processor 20 (i.e., main processing unit 150 and/or interlock processing unit 152). As stated above, dispenser assembly 12 includes a fluid reservoir 14 having a plunger (not shown) that moves axially, such that the direction of movement of the plunger is dependant upon whether the fluid reservoir 14 is being filled or emptied. Prior to use of infusion pump 10, fluid reservoir 14 must be filled with medicament, as it is typically shipped from the factory empty.

Prior to filling fluid reservoir 14 of dispenser assembly 12 with medicament, infusion pump 10 is in an inactive state, thereby reducing power consumption and lengthening shelf life. When it is time to use infusion pump 10, the patient must fill the fluid reservoir 14 of dispenser assembly 12 with medicament. Once fluid reservoir 14 is filled with at least a predefined volume of medicament (e.g., 50 units), the plunger of the fluid reservoir 14 contacts fill sensor 24, thereby providing the initialization signal to local processor 20. At this point, the various components of infusion pump 10 are initialized and begin to operate as described above.

A number of implementations have been described. Nevertheless, it will be understood that various modifications may be made. Accordingly, other implementations are within the scope of the following claims.

1. A system for delivering a fluid to a patient comprising:
a remote controller; and
an infusion pump including:
a dispenser for dispensing the fluid;
an RF telemetry portion configured to receive an RF data signal, from the remote controller, that is broadcast in a non-restricted frequency band;
a processing portion configured to process the RF data signal received by the RF telemetry portion, and control the dispenser in accordance with the RF data signal received by the RF telemetry portion.

2. The system of claim 1 wherein the non-restricted frequency band is 13.40-13.70 megahertz.

3. The system of claim 1 wherein the RF telemetry portion is further configured to receive data encoded within a 13.56 megahertz carrier signal.

4. The system of claim 1 wherein the RF telemetry portion is further configured to transmit data encoded within a 13.56 megahertz carrier signal.

5. A medical device comprising:
an RF telemetry portion configured to receive an RF data signal that is broadcast in a non-restricted frequency band; and
a processing portion configured to process the RF data signal received by the RF telemetry portion.

6. The medical device of claim 5 wherein the processing portion includes:
a main processing unit; and
an interlock processing unit.

7. The medical device of claim 5 wherein the non-restricted frequency band is 13.40-13.70 megahertz.

8. The medical device of claim 5 wherein the RF telemetry portion is further configured to receive data encoded within a 13.56 megahertz carrier signal.

9. The medical device of claim 5 wherein the RF telemetry portion is further configured to transmit data encoded within a 13.56 megahertz carrier signal.

10. The medical device of claim 5 wherein the RF telemetry portion includes compact antenna.

11. The medical device of claim 10 wherein the compact antenna is a spirally-wound antenna.

12. The medical device of claim 10 wherein the compact antenna is a helically-wound antenna.

13. The medical device of claim 10 wherein an effective length of the compact antenna is a defined percentage of a wavelength of a carrier signal.

14. The medical device of claim 5 wherein the RF data signal includes a defined validation sequence and the RF telemetry portion is further configured to:
examine the RF data signal to confirm that the RF data signal includes the defined validation sequence.

15. The medical device of claim 14 wherein the RF telemetry portion is further configured to:
transmit an acknowledgement signal to the device transmitting the RF data signal if it is determined that the RF data signal includes the defined validation sequence.

16. The medical device of claim 5 wherein the RF telemetry portion and at least a first portion of the processing portion are incorporated into a single microchip.

17. (canceled)

18. The medical device of claim 5 further comprising:
a dispensing apparatus, responsive to the processing portion of the medical device, for dispensing medicament in accordance with the RF data signal.

19. The medical device of claim 18 wherein the medicament is insulin.

20. A method of communicating with a medical device comprising:
receiving an RF data signal that is broadcast in a non-restricted frequency band; and
processing the RF data signal received.

21. The method of claim 20 wherein the non-restricted frequency band is 13.40-13.70 megahertz.

22. The method of claim 20 wherein receiving an RF data signal includes:
receiving data encoded within a 13.56 megahertz carrier signal.

23. The method of claim 20 further comprising:
transmitting data encoded within a 13.56 megahertz carrier signal.

24. The method of claim 20 wherein the RF data signal includes a defined validation sequence, the method further comprising:
examining the RF data signal to confirm that the RF data signal includes the defined validation sequence.

25. The method of claim 24 further comprising:
transmitting an acknowledgement signal to the device transmitting the RF data signal if it is determined that the RF data signal includes the defined validation sequence.

26. A computer program product residing on a computer readable medium having a plurality of instructions stored thereon which, when executed by the processor, cause that processor to:
receive an RF data signal that is broadcast in a non-restricted frequency band; and
process the RF data signal received.

27. The computer program product of claim 26 wherein the non-restricted frequency band is 13.40-13.70 megahertz.

28. The computer program product of claim 26 wherein the instructions for receiving an RF data signal include instructions for:
receiving data encoded within a 13.56 megahertz carrier signal.

29. The computer program product of claim 26 further comprising instructions for:
transmitting data encoded within a 13.56 megahertz carrier signal.

30. The computer program product of claim 26 wherein the RF data signal includes a defined validation sequence, the computer program product further comprising instructions for:
examining the RF data signal to confirm that the RF data signal includes the defined validation sequence.

31. The computer program product of claim 30 further comprising instructions for:
transmitting an acknowledgement signal to the device transmitting the RF data signal if it is determined that the RP data signal includes the defined validation sequence.
